(19) United States
(12) Patent Application Publication
(10) Pub. No.: US 2004/0241092 A1
(43) Pub. Date: Dec. 2, 2004

(54) Method for determining alleviation of pain in an animal

(76) Inventors: Suzan Elizabeth Bove, Ann Arbor, MI (US); Kenneth Kilgore, Ann Arbor, MI (US)

Correspondence Address: WARNER-LAMBERT COMPANY, 2800 PLYMOUTH RD, ANN ARBOR, MI, 48105, US

(21) Appl. No.: 10/856,673

(22) Filed: May 28, 2004

Related U.S. Application Data
(60) Provisional application No. 60/474,658, filed on May 30, 2003.

(51) Int. Cl. A61K 49/00

(52) U.S. Cl. 424/9.2; 424/85.2

(57) ABSTRACT

This invention relates to a method for determining alleviation by a test compound of pain in an animal, a method for screening potential analgesic compounds in vivo for effectiveness of alleviating pain, a method of inducing pain in a pharmacological study animal that closely mimics a pain of a human or animal disease, and a method for characterizing a known analgesic for its pain alleviating activity in the human or animal disease.

### CROSS-REFERENCE TO RELATED APPLICATIONS

This application claims benefit from U.S. Provisional Patent Application No. 60/474,658, filed May 30, 2003.

### FIELD OF THE INVENTION

This invention relates to a method for determining alleviation by a test compound of pain in an animal, a method for screening potential analgesic compounds in vivo for effectiveness of alleviating pain, a method of inducing pain in a pharmacological study animal that closely mimics a pain of a human or animal disease, and a method for characterizing a known analgesic for its pain alleviating activity in the human or animal disease.

### BACKGROUND OF THE INVENTION

It has been estimated that more than 50 million people, including 23 million Americans, have some form of arthritis, a chronic disease for which there is no known cure. Among the various forms of arthritis, osteoarthritis (“OA”) is the most prevalent, afflicting 21 million people in the United States alone. Rheumatoid arthritis (“RA”) afflicts an additional 2.5 million people in the United States. One common symptom of arthritis sufferers is joint pain.

Aspirin and conventional nonsteroidal anti-inflammatory drugs (NSAIDs) such as ibuprofen, diclofenac, and naproxen are the primary agents used to treat arthritic pain. However, the long-term therapeutic use of conventional NSAIDs is limited due to drug associated side effects, including life threatening gastric ulceration due to suppression of the gastrointestinal mucosa, thromboembolic liability and suppression of platelet aggregation, and cardiorenal toxicities such as edema, impairment of renal function, elevated blood pressure, and effects on glomerular filtration rate. Even cyclooxygenase-2 (“COX-2”) inhibitors show some of these toxicities, albeit their safety profiles are improved versus nonselective inhibitors of the cyclooxygenases.

The need for new therapies for treating arthritic pain continues. The present invention provides a method for screening test compounds or characterizing known analgesic compounds for arthritic pain alleviating activity. The invention is also a new animal model that closely mimics arthritic pain. All that is required to practice the invention is to screen or characterize a compound according to the methods described below.

### SUMMARY OF THE INVENTION

As mentioned previously, this invention relates to a method for determining alleviation by a test compound of pain in an animal, a method for screening potential analgesic compounds in vivo for effectiveness of alleviating pain, a method of inducing pain in a pharmacological study animal that closely mimics a pain of a human or animal disease, and a method for characterizing a known analgesic for its pain alleviating activity in the human or animal disease.

In a first aspect, this invention is a method for determining alleviation by a test compound of pain in an animal, comprising:

administering a measured amount of a test compound dissolved in a test compound vehicle to each of a group of from 1 to 500 treatment animals;

administering to a hind leg knee joint of each of the treatment animals a pain inducing amount of IL-6 dissolved in a cytokine vehicle, IL-6sR dissolved in a cytokine vehicle, or of a combination of IL-6 and IL-6sR dissolved in a cytokine vehicle;

characterizing pain in the hind leg knee joint of each of the treatment animals after the administration of IL-6 dissolved in a cytokine vehicle, IL-6sR dissolved in a cytokine vehicle, or of a combination of IL-6 and IL-6sR dissolved in a cytokine vehicle by measuring differential hind paw weight distribution; and

comparing the differential hind paw weight distribution measurements for the from 1 to 500 treatment animals to a control value.

Another aspect of this invention is the method according to the first aspect, further comprising the step of deriving the control value by:

administering to a hind leg knee joint of each of a group of from 1 to 500 control animals a pain inducing amount of IL-6 dissolved in a cytokine vehicle, IL-6sR dissolved in a cytokine vehicle, or of a combination of IL-6 and IL-6sR dissolved in a cytokine vehicle; and

characterizing pain in the hind leg knee joint of each of the control animals by measuring differential hind paw weight distribution.

Another aspect of this invention is the method according to the first aspect, further comprising the step of deriving the control value by:

administering an amount of a test compound vehicle to each of a group of from 1 to 500 control animals;

administering to a hind leg knee joint of each of the control animals a pain inducing amount of IL-6 dissolved in a cytokine vehicle, IL-6sR dissolved in a cytokine vehicle, or of a combination of IL-6 and IL-6sR dissolved in a cytokine vehicle; and

characterizing pain in the hind leg knee joint of each of the control animals by measuring differential hind paw weight distribution.

Another aspect of this invention is an above method, wherein the control value is derived before the test compound dissolved in test compound vehicle and the IL-6 dissolved in a cytokine vehicle, IL-6sR dissolved in a cytokine vehicle, or of the combination of IL-6 and IL-6sR dissolved in a cytokine vehicle are administered.

Another aspect of this invention is an above method, wherein the control value is derived before the comparison of the differential hind paw weight distribution measurements.

Another aspect of this invention is a method for determining alleviation by a test compound of pain in an animal, comprising:

administering an amount of a test compound vehicle to each of a group of from 1 to 500 control animals;

administering a measured amount of the test compound dissolved in a test compound vehicle to each of a group of from 1 to 500 treatment animals;

administering to a hind leg knee joint of each of the control animals and treatment animals a pain inducing amount of IL-6 dissolved in a cytokine vehicle, IL-6sR dissolved in a cytokine vehicle, or of a combination of IL-6 and IL-6sR dissolved in a cytokine vehicle;

characterizing pain in the hind leg knee joint of each of the control animals and treatment animals by measuring differential hind paw weight distribution; and

characterizing the differential of pain in the hind leg knee joints of the control animals and pain in the hind leg knee joints of the treatment animals.

Another aspect of this invention is a method for determining alleviation by a test compound of pain in an animal, comprising:

administering a measured amount of a test compound vehicle to each of a group of from 1 to 500 control animals;

administering a measured amount of the test compound dissolved in a test compound vehicle to each of a group of from 1 to 500 treatment animals;

administering to a first hind leg knee joint of each of the control animals and treatment animals a pain inducing amount of IL-6 dissolved in a cytokine vehicle, IL-6sR dissolved in a cytokine vehicle, or a combination of IL-6 and IL-6sR dissolved in a cytokine vehicle;

administering to a second hind leg knee joint of each of the control animals and treatment animals a measured amount of cytokine vehicle;

characterizing pain in the first hind leg knee joint of each of the control animals and treatment animals by measuring differential hind paw weight distribution; and

characterizing the differential of pain in the first hind leg knee joints of the control animals and pain in the first hind leg knee joints of the treatment animals.

Another aspect of this invention is any one of the above methods, wherein the number of control animals is, increasingly preferably, from 3 to 250, from 3 to 100, from 5 to 50, from 5 to 20, from 5 to 15, from 7 to 10, 10, 9, 8, 7, 6, or 5.

Another aspect of this invention is any one of the above methods, wherein the number of treatment animals is, increasingly preferably, from 3 to 250, from 3 to 100, from 5 to 50, from 5 to 20, from 5 to 15, from 7 to 10, 10, 9, 8, 7, 6, or 5.

Another aspect of this invention is any one of the above methods, wherein the compound vehicle comprises HPMC, Tween 80, saline, or water.

Another aspect of this invention is any one of the above methods, wherein the cytokine vehicle comprises PBS or saline.

Another aspect of this invention is any one of the above methods, wherein a combination of IL-6 and IL-6sR is administered.

Another aspect of this invention is any one of the above methods, wherein a solution of a combination of IL-6 and IL-6sR in the cytokine vehicle is administered.

Another aspect of this invention is any one of the above methods, wherein the IL-6 and IL-6sR are separately dissolved in cytokine vehicle and administered sequentially.

Another aspect of this invention is any one of the above methods, wherein IL-6 is administered.

Another aspect of this invention is any one of the above methods, wherein IL-6sR is administered.

Another aspect of this invention is any one of the above methods, wherein the IL-6, IL-6sR, or IL-6 and IL-6sR is a human form.

Another aspect of this invention is any one of the above methods, wherein the IL-6, IL-6sR, or IL-6 and IL-6sR is a recombinant human form.

Another aspect of this invention is any one of the above methods, wherein the animal is a rat, mouse, or rabbit.

Another aspect of this invention is any one of the above methods, wherein the animal is a rat.

Another aspect of this invention is any one of the above methods, wherein the animal is a mouse.

Another aspect of this invention is any one of the above methods, wherein the animal is a rabbit.

Another aspect of this invention is any one of the above methods, wherein the test compound is a known analgesic.

Another aspect of this invention is any one of the above methods, wherein the test compound is not a known analgesic.

Another aspect of this invention is any one of the above methods, wherein the test compound is a small organic molecule of from 100 to 1000 atomic mass units, preferably from 200 to 600 atomic mass units.

Another aspect of this invention is any one of the above methods, wherein the test compound is a biological therapeutic agent, preferably an anti-tumor necrosis factor-alpha monoclonal antibody or anti-tumor necrosis factor-alpha receptor immunoglobulin molecule.

Another aspect of this invention is any one of the above methods, wherein the differential of pain is qualitatively characterized.

Another aspect of this invention is any one of the above methods, wherein the differential of pain is quantitatively characterized.

Another aspect of this invention is any one of the above methods, wherein the test compound is administered orally.

Another aspect of this invention is any one of the above methods, wherein the test compound is administered orally before administration of the IL-6, IL-6sR, or IL-6 and IL-6sR.

Another aspect of this invention is any one of the above methods, wherein the test compound is administered intravenously.

Another aspect of this invention is any one of the above methods, wherein the test compound is administered subcutaneously.

Another aspect of this invention is any one of the above methods, wherein the test compound is administered intraperitoneally.

Another aspect of this invention is any one of the above methods, wherein the test compound is administered topically.

Another aspect of this invention is any one of the above methods, wherein the test compound is administered concurrently with administration of the IL-6, IL-6sR, or IL-6 and IL-6sR.

Another aspect of this invention is any one of the above methods, wherein the test compound is administered before administration of the IL-6, IL-6sR, or IL-6 and IL-6sR.

Another aspect of this invention is any one of the above methods, wherein the test compound is administered intravenously after administration of the IL-6, IL-6sR, or IL-6 and IL-6sR.

Another aspect of this invention is any one of the above methods, wherein the administration of the IL-6, IL-6sR, or IL-6 and IL-6sR is carried out, increasingly preferably, from 0 minutes to 8, 0 to 4, 0 to 2, 0.5 to 2, 1 to 2, or 0.8 to 1.2 hours after administration of test compound.

Another aspect of this invention is any one of the above methods, wherein the administration of the IL-6, IL-6sR, or IL-6 and IL-6sR is carried out, increasingly preferably, from 0.5 to 2, 0.8 to 1.5, 0.8 to 1.2, or 0.9 to 1.1 hours before a time of peak plasma concentration of the administered test compound.

Another aspect of this invention is any one of the above methods, wherein the method comprises a dose response characterization of the differential of pain for the test compound administered in from 3 to 20 different measured amounts.

Another aspect of this invention is any one of the above methods, wherein method comprises a time course characterization of the differential of pain for the test compound administered at one measured amount, wherein the time course comprises measurements taken at from 3 to 20 different time points.

Another aspect of this invention is a method for determining alleviation, by a test combination, of (IL-6)-, (IL-6sR)-, or (IL-6 plus IL-6sR)-induced pain in an animal, comprising:

administering a measured amount of a test combination dissolved in a test compound vehicle to each of a group of from 1 to 500 treatment animals;

administering to a hind leg knee joint of each of the treatment animals a pain inducing amount of IL-6 dissolved in a cytokine vehicle, IL-6sR dissolved in a cytokine vehicle, or of a combination of IL-6 and IL-6sR dissolved in a cytokine vehicle;

characterizing pain in the hind leg knee joint of each of the treatment animals after the administration of IL-6 dissolved in a cytokine vehicle, IL-6sR dissolved in a cytokine vehicle, or of a combination of IL-6 and IL-6sR dissolved in a cytokine vehicle by measuring differential hind paw weight distribution; and

comparing the differential hind paw weight distribution measurements for the from 1 to 500 treatment animals to a control value.

Another aspect of this invention any one of the above methods, wherein “test combination” is employed in place of “test compound” and “test combination vehicle” is employed in place of “test compound vehicle.”

Another aspect of this invention is any one of the above methods, wherein the compound or combination is being tested for alleviating pain associated with systemic lupus erythematosus, mixed connective tissue disease, or arthritic diseases such as rheumatoid arthritis, osteoarthritis, gouty arthritis, juvenile arthritis, ankylosing spondylitis, and reactive arthritis (i.e., Reiter's arthritis).

Another aspect of this invention is any one of the above methods, wherein the IL-6, IL-6sR, or IL-6 and IL-6sR is replaced by a protein or protein and its receptor selected from: oncostatin-M, oncostatin-M and oncostatin-M receptor, leukemia inhibitor factor (“LIF”), LIF and leukemia inhibitor factor receptor (“LIFR”), interleukin-11 (“IL-11”), and IL-11 and interleukin-11 receptor (“IL-11R”) and the cytokine vehicle is replaced by protein vehicle.

### BRIEF DESCRIPTION OF THE DRAWINGS

FIG. 1 (“FIG. 1”) is a line graph that shows the effect on rat hind paw weight distribution, expressed in grams, of an intra-articular injection through the patellar ligament into the joint space of certain amounts (10, 30, 100, or 300 ng) of IL-6 dissolved in 50 μL of PBS versus injection of 50 μL of PBS alone.

FIG. 2 (“FIG. 2”) is a line graph that shows the effect on rat hind paw weight distribution, expressed in grams, of an intra-articular injection through the patellar ligament into the joint space of a combination of 100 ng IL-6 and certain amounts of IL-6sR (100, 300, or 1000 ng) dissolved in 50 μL of PBS versus injection of 50 μL of PBS alone.

FIG. 3 (“FIG. 3”) is a bar graph that shows the effect on rat hind paw weight distribution, expressed in grams, before (Time 0 hour) and after (Time 1 hour) an intra-articular injection through the patellar ligament into the joint space of a combination of 100 ng of IL-6 and 300 ng of IL-6sR, 100 ng of IL-6 alone, 300 ng of IL-6sR alone, or a combination of 100 ng of heat inactivated IL-6 and 300 ng of IL-6sR, each dissolved in 50 μL of PBS.

FIG. 4 (“FIG. 4”) is a dose response line graph that shows the percent inhibition of a change in rat hind paw weight distribution following oral administration at Time 0 of 1, 3, 10, and 30 mg/kg of naproxen or rofecoxib dissolved in HPMC, followed by an intra-articular injection through the patellar ligament into the joint space at Time 1 hour of a combination of 100 ng IL-6 and 300 ng of IL-6sR dissolved in 50 μL of PBS, followed by hind paw weight distribution measurements taken at Time 2 hours (1 hour post injection), compared to a control group for oral administration of HPMC/TWEEN 80 alone.

FIG. 5 (“FIG. 5”) is a time course line graph that shows the change in rat hind paw weight distribution, expressed in grams, following oral administration at Time 0 of 10 mg/kg of naproxen or 10 mg/kg of rofecoxib dissolved in HPMC/TWEEN 80 and an intra-articular injection through the patellar ligament into the joint space at Time 1 hour of a combination of 100 ng IL-6 and 300 ng of IL-6sR dissolved in 50 μL of PBS, compared to a control group for oral administration of HPMC/TWEEN 80 alone. The rat hind paw weight distribution data were measured at Time 0, 1, 3, and 6 hours post injection or 1, 2, 4, and 7 hours post administration, respectively.

### DETAILED DESCRIPTION OF THE INVENTION

This invention relates to a method for determining alleviation by a test compound of pain in an animal, a method for screening potential analgesic compounds in vivo for effectiveness of alleviating pain, a method of inducing pain in a pharmacological study animal that closely mimics a pain of a human or animal disease, and a method for characterizing a known analgesic for its pain alleviating activity in the human or animal disease.

A test compound in the method of the present invention may be administered in combination with other therapeutic agents, including known analgesic agents, to determine analgesic effects, including synergistic analgesic effects, wherein the therapeutic agents may be a small organic molecule, as defined herein, or a biological therapeutic, as defined herein. A combination administered in a method of the present invention may be referred to herein as a “test combination.”

The invention method is useful in human and veterinary medicines for identifying new arthritic pain alleviating agents, for characterizing the arthritic pain alleviating effects of known analgesics, and for finding new medical uses for known compounds. Arthritic pain alleviating effects of biological therapeutics useful for treating arthritic conditions, including CP-870, etanercept (a tumor necrosis factor alpha (“TNF-alpha”) receptor immunoglobulin molecule; trade names ENBREL® and ENBREL ENTANERCEPT® by Immunex Corporation, Seattle, Wash.), infliximab (an anti-TNF-alpha chimeric IgG. 1K monoclonal antibody; tradename REMICADE® by Centocor, Inc., Malvern, Pa.), methotrexate (tradename RHEUMATREX® by American Cyanamid Company, Wayne, N.J.), and adalimumab (a human monoclonal anti-TNF-alpha antibody; tradename HUMIRA® by Abbott Laboratories, Abbott Park, Ill.) may also be characterized according to the present invention.

It should be appreciated that the number of control animals and treatment animals used in a method of the present invention may be the same or different.

It should also be appreciated that the cytokine vehicle used in the control and treatment animal groups in a method of the present invention may be the same or different and that the test compound vehicle used in the control and treatment animal groups in a method of the present invention may be the same or different. Further, the cytokine vehicle may be the same or different as the compound vehicle.

It should also be appreciated that characterizing hind paw weight bearing differentials may be carried out using an incapacitance tester, which is described below. However, any method of measuring hind paw weight distribution in an animal may be used in a method of the present invention.

The terms and phrases used herein are as defined below or as they otherwise occur in the specification.

HPMC means hydroxymethylpropylcellulose.

PBS means phosphate buffered saline.

The term “alleviation” as it applies herein to an effect on pain means the effect of suppressing, reducing, preventing, or otherwise inhibiting pain or discomfort in an animal, including, but not limited to, the suppression, reduction, prevention, or inhibition of (IL-6)-, (IL-6sR)-, or (IL-6 plus IL-6sR)-induced pain symptoms of a mechanical or inflammatory nature.

The term “pain” means discomfort or pain of a joint or a nearby tissue, including nearby bone or muscle. Such pain may manifest itself in an animal by, for example, altering the gait, reducing freedom of joint movement, altering weight distribution of the animal, or increasing sensitivity to one or more stimuli such as heat or mechanical pressure in the animal.

The phrase “test compound” means a compound, a pharmaceutically acceptable salt thereof, a prodrug thereof, a radiolabeled version thereof, or the like. A test compound may be a small (i.e., molecular weight of from 100 to 1000 atomic mass units (“AU”), preferably from 200 to 600 AU) organic molecule or a biological therapeutic such as a monoclonal antibody, soluble receptor, receptor ligand, anti-sense ribonucleic acid (“RNA”), deoxyribonucleic acid (“DNA”), and the like.

The phrase “test combination” means a combination of two test compounds as defined herein.

It should be appreciated that “rofecoxib” is a COX-2 inhibitor. A formulated version of rofecoxib is marketed under the tradename VIOXX® by MERCK & CO., Inc., Whitehouse Station, N.J.

The phrase “(IL-6)-, (IL-6sR)-, or (IL-6 plus IL-6sR)-induced pain” means pain that is, directly or indirectly, the result of, triggered by, or follows from administration of IL-6, IL-6sR, or a combination of IL-6 plus IL-6sR, respectively, to an animal, including administration to a joint in the animal.

The phrase “test compound vehicle” means any liquid carrier or diluent that is chemically compatible with a test compound or test combination of a test compound and another therapeutic agent and is capable of dissolving or finely suspending the test compound to give a concentration of test compound or test combination of greater than or equal to 1% weight/volume.

The phrase “test combination vehicle” means any liquid carrier or diluent that is chemically compatible with a test compound or test combination and is capable of dissolving or finely suspending the test compound or test combination to give a total concentration of test compound or test combination of greater than or equal to 1% weight/volume. The test combination vehicle may be the same or different for each component of a test combination, and a solution of each component of the test combination may be combined to make one solution for administration or kept separate for administration.

The phrase “cytokine vehicle” means any liquid carrier or diluent that is chemically and biochemically compatible with IL-6, IL-6sR, or a combination of IL-6 and IL-6sR and is capable of dissolving or finely suspending the IL-6, IL-6sR, or a combination of IL-6 and IL-6sR to give a concentration of IL-6, IL-6sR, or a combination of IL-6 and IL-6sR of greater than or equal to 0.1% weight/volume.

The phrase “protein vehicle” means any liquid carrier or diluent that is chemically and biochemically compatible with oncostatin-M, oncostatin-M and oncostatin-M receptor, LIF, LIF and LIFR, IL-11, or IL-11 and IL-11R. Illustrative examples of protein vehicle include PBS, HPMC, HPMC with Tween-80, and the like.

The term “dissolved” as applied to a test compound or test combination means that the test compound or test combination is fully dissolved in test compound solution or is partially dissolved in test compound solution, wherein partially dissolved in test compound solution means from 99.9% to 75% of the test compound or test combination is dissolved in test compound solution. The term “dissolved” as applied to IL-6, IL-6sR, or a combination of IL-6 and IL-6sR means that the IL-6, IL-6sR, or a combination of IL-6 and IL-6sR is at least 50%, increasingly preferably at least 60%, at least 67%, at least 75%, at least 88%, at least 94%, at least 97%, at least 98%, at least 99%, or 100% dissolved in cytokine solution.

It should be appreciated that a useful amount of a test compound vehicle or a cytokine vehicle may be readily and independently determined by an artisan in the pharmacology art by routine solubility experimentation. Such amounts will be from the minimum amount necessary to fully dissolve or finely suspend the test compound or cytokine, respectively, up to from about 1.1 times to about 10 times the minimum amount.

The term “comparing” means examining two or more pieces of data and ascertaining qualitative or quantitative similarities or differences.

The phrase “control value” means a baseline value for a control animal, or an average or other statistical grouping of baseline values for two or more control animals, of the measurement being compared. For example, a control value for a differential hind paw weight distribution measurement is the baseline value or values of a hind paw weight distribution measurement for a control animal or animals, respectively.

The phrase “control animal” means an animal to which a test compound or test combination is not administered in a method of the present invention. A control animal may also be an animal to which a test compound or test combination is not administered, although test compound vehicle may or may not be administered to the animal, wherein the control animal is separately used for the purposes of determining a control value for the method of the present invention

The phrase “treatment animal” means an animal to which a test compound is administered in an invention method.

It should be appreciated that a control or treatment animal means the control or treatment animal, respectively, that is participating in a method of the present invention. An animal that is not administered a test compound or test combination is not a treatment animal. An animal that is not administered IL-6, IL-6sR, or a combination of IL-6 and IL-6sR is not a control or treatment animal.

The phrase “hind leg knee joint” means the knee joint (as opposed to the ankle or hip joints) of a hind (i.e., rear or back) leg of an animal.

The phrase “hind paw” means the paw of a hind (i.e., rear or back) leg of an animal.

The phrase “pain inducing amount of IL-6” means an amount of IL-6 that is sufficient to induce a quantitatively measurable or qualitatively discernable differential of pain, as determined by ascertaining a differential hind paw weight distribution with an incapacitance tester, following administration thereof. Similarly, a pain-inducing amount of IL-6sR or a combination of IL-6 and IL-6sR means an amount of IL-6sR or of the combination sufficient to induce a quantitatively measurable or qualitatively discernable differential of pain, as determined by ascertaining a differential hind paw weight distribution with an incapacitance tester, following administration thereof.

It should be appreciated that pain inducing amounts of IL-6, IL-6sR, or a combination of IL-6 and IL-6sR are readily determined by an artisan in the pharmacology art by routine experimentation. In a rat, for example, one aspect of this invention is an amount of IL-6 of from about 10 nanograms (“ng”) to about 500 ng, or about 100 ng, and an amount of IL-6sR of from about 100 ng to about 1000 ng, or about 300 ng.

Interleukin-6 and interleukin-6 soluble receptor mean Il-6 and IL-6sR, respectively, that is derived from a natural source such as a human, rat, mouse, rabbit, guinea pig, monkey, dog, cat, pig, cow, horse, and the like, and recombinant versions thereof, and active truncated versions thereof. In one aspect of this invention human, rat, mouse, or rabbit IL-6 or IL-6sR is used. In another aspect, recombinant human IL-6 (“rhIL-6”) or recombinant human IL-6sR (rhIL-6sR”) is used. For the purposes of this invention, it does not matter which of the above-identified forms of IL-6 or IL-6sR are used. Further, a mixture of IL-6 or a mixture of IL-6sR, each from more than one source (an animal, recombinant, active truncated versions) may be used in a method of this invention.

It should be appreciated that any protein or protein and its receptor that interacts directly or indirectly with a GP130 receptor may be used in an invention method in place of Il-6 and/or IL-6sR. The protein or the protein and its receptor may be selected from: oncostatin-M, oncostatin-M and oncostatin-M receptor, LIF, LIF and LIFR, IL-11, and IL-11 and IL-11R may be used in an invention method in place of Il-6 and/or IL-6sR.

The phrase “differential hind paw weight distribution” means the difference between the body weight of an animal on its left hind paw and the body weight of the animal on its right hind paw, expressed in a unit of mass, preferably in grams. One method, which is not necessarily the only method, that may be used to determine differential hind paw weight distribution in an animal is to measure the amount of the animal's body weight on the animal's hind paws using an incapacitance tester, which is described below.

The phrase “characterizing pain in the hind leg knee joint” refers to determining the differential in weight bearing on an animal's hind paws such as, for example, by placing an animal on a tray having a floor with holes for weighing an animals hind paws and a forward-sloping wall to support the animal's front, and measuring the difference in weight on the animal's left hind paw versus its right hind paw, such as described below in Method Example 1.

The phrase “characterizing the differential of pain” as applied above to the pain in the hind leg knee joints of the control animals and the pain in the hind leg knee joints of the treatment animals means averaging or otherwise mathematically combining the individual animal's differential in weight bearing on the animal's hind paws in the control animal group and separately in the treatment animal group as described above and below, and ascertaining any difference in pain between the animal groups. Such difference can be statistically analyzed using, for example, an analysis of covariance (“ANCOVA”) followed by Hochberg's procedure.

The phrase “dose response” means a method wherein test compound is administered at various doses to different treatment animals. In one aspect of this invention, the doses for a dose response method of the instant invention are 0.3, 1, 3, and 10 mg/kg; 1, 3, 10, and 30 mg/kg; and 3, 10, 30, 100 mg/kg.

The phrase “time course” means a method wherein test compound is administered at a single dose to a treatment animal and pain is characterized by hind paw weight distribution measurements taken at different time points. In one aspect of this invention, the time points are 0, 0.5, 1, and 3 hours; 0, 1, 3, and 6 hours; and 0, 3, 6, and 12 hours post injection of IL-6, IL-6sR, or a combination of IL-6 and IL-6sR.

For the purposes of this invention, the term “arthritis” includes osteoarthritis, rheumatoid arthritis, degenerative joint disease, spondyloarthropathies, gouty arthritis, systemic lupus erythematosus, mixed connective tissue disease, juvenile arthritis, reactive arthritis, and psoriatic arthritis.

It should be appreciated that the phrases “invention method,” “present invention method,” “method of the present invention,” “method of the invention,” and the like are synonymous.

In determining what constitutes a proper amount of a test compound to administer according to the invention method, a number of factors will generally be considered by one skilled in the pharmacological art, including the species of animal used, the weight of the animal used, the route of administration (e.g., oral, intravenous, topical, etc.), the mode of administration (e.g., quaque die (“QD,” meaning once daily) bis in die (“BID,” meaning twice daily), ter in die (“TID,” meaning three times a day), or quater in die (“QID,” meaning four times a day), etc.), whether a single dose or a dose response experimental paradigm is being employed, the compound vehicle being employed, the class of compound tested (e.g., COX-2 inhibitor, alpha-2-delta ligand, opioid, etc.), the potency of the test compound in its class, the bioavailability of the test compound in the particular species, and pharmacokinetic and pharmacodynamic profile, including half-life, area under the concentration curve, etc. of the test compound, and the like. Such amounts will generally be from about 0.1 mg/kg to about 300 mg/kg of animal weight. Typical doses will be from about 10 to about 100 mg/kg of animal weight.

As such, the administered dose may fall within the ranges or amounts recited above, or may vary outside, ie, either below or above, those ranges depending upon the requirements of the particular experiment.

The invention method may be conducted by administering a test compound either alone or formulated in a composition suitable for pharmaceutical administration. Pharmaceutical compositions are produced by formulating the active compound in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses.

Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the invention method can also contain other components such as coloring agents, flavoring agents, and/or preservatives. These materials, if present, are usually used in relatively small amounts. The compositions can, if desired, also contain other therapeutic agents commonly employed to treat osteoarthritis. Further, the compositions can, if desired, also contain other therapeutic agents commonly employed to treat secondary symptoms such as, for example, inflammation or pain that may or may not accompany cartilage damage. For example, the compositions may contain aspirin, naproxen, or similar anti-inflammatory analgesic agents.

The percentage of the active ingredients in the foregoing compositions can be varied within wide limits, but for practical purposes it is preferably present in a concentration of at least 10% in a solid composition and at least 2% in a primary liquid composition. The most satisfactory compositions are those in which a much higher proportion of the active ingredient is present, for example, up to about 95%.

Preferred routes of administration of a test compound are oral, subcutaneous, intraperitoneally, or parenteral. For example, a useful intravenous dose is between 5 and 50 mg/kg of animal body weight, and a useful oral dosage is between 20 and 100 mg/kg. The dosage is within the dosing range used in treatment of diseases resulting in arthritic pain as described above.

The advantages of the instant invention method include the fact that it facilitates rapid in vivo testing of compounds for analgesic activity. Typical in vivo methods for ascertaining arthritic pain in a joint take many hours (e.g., carageenan induced thermal hyperalgesia (“CITH”) in rat) and in some cases days (e.g., monosodium iodoacetate-induced osteoarthritis (“MIA”) in rat takes at least 7 days to provide a pain readout). The instant invention method provides results in one hour, or less, post administration of IL-6 or a combination of IL-6 and IL-6sR.

The invention method is also reliable, providing reproducible results, and flexible, capable of rapidly providing dose-response data in one hour or less, if desired, and of providing time course studies of more than 6 hours in duration post administration of IL-6 or a combination of IL-6 and IL-6sR.

Another advantage of the instant invention method is that it induces joint pain without complications from pathophysiological damage to the joint such as the cartilage damage that accompanies the MIA model.

Another advantage of the instant invention is that it more closely mimics arthritic joint pain than a thermal hyperalgesia model of pain. Osteoarthritic pain is not a hyperalgesia pain, but instead a mechanical pathophysiological pain similar to the pain induced in the instant invention method. The present (IL-6)-, (IL-6sR)-, and (IL-6 plus IL-6sR)-induced pain rat, mouse, or rabbit models described herein are predictive of pain in a human or other mammals such as dog, cat, pig, cow, horse, monkey, chimpanzee, rat, mouse, rabbit, guinea pig, hamster, sheep, goat, and the like by virtue of the fact that rat, mouse, rabbit, human, and the other mammals have IL-6, IL-6sR, and bound IL-6 receptor in joints. Further, both humans with arthritis of a knee or hip joint and animals with (IL-6)-, (IL-6sR)-, and (IL-6 plus IL-6sR)-induced knee or hip joint pain have been observed to exhibit an altered gait (walk), whereas an altered gait is not observed for animals in a thermal hyperalgesia model of pain. This is why pain models such as footpad thermal hyperalgesia models, which are not joint based and are heat sensitive as opposed to mechanically sensitive, are less predictive of osteoarthritic pain alleviation in a human or other mammal than the instant pain model.

Without wishing to be bound by a theory, the inventors believe that the instant model is a mechanical pathophysiological model of pain wherein there is a direct effect on nerve cells of the joint synovium, nearby bone, or musculature. It is known that bound IL-6 receptor is found in synovium and bone (osteoblast cells) of mammals and that it is cleaved in vivo to form a soluble fragment that is its soluble receptor or IL-6R. Cytokine IL-6 and its soluble receptor IL-6sR may found in extracellular fluids such as synovial fluid in mammals. The inventors believe that, as demonstrated below, injection of IL-6sR alone or a combination of heat inactivated IL-6 and IL-6sR induces pain in an animal according to this invention by combining with endogenous IL-6 of the animal.

It should be appreciated that in all of the below examples, unless indicated otherwise, male Wistar rats (175-200 g) were housed in solid bottom isolator cages, 2-4 rats per cage, with corncob bedding on a 12 hour:12 hour light:dark cycle. Animals were fed standard rat chow with water available ad libitum.

It should be appreciated that in all of the below examples, hind paw weight bearing differentials between the paw of the limb having the control joint (in animals administered test compound vehicle only) and the paw of the limb having the contralateral test joint (in control or inducement animals administered test compound vehicle only and in treatment animals administered test compound dissolved in test compound vehicle) were determined using an incapacitance tester, model 2KG (Linton Instrumentation, Norfolk, United Kingdom). The incapacitance tester has a chamber on top with an outwardly sloping front wall that supports a rat's front limbs, and two weight sensing pads, one for each hind paw, that facilitates this determination. Each data point is the mean of three readings of 5 seconds duration. The hind paw weight bearing differentials were expressed in grams and calculated as the [sum of (the weight in grams placed on the control limb minus the weight in grams placed on the contralateral test limb for each animal in the inducement or treatment animal group)] divided by the number of animals in the inducement or treatment animal group. The results of these determinations may be displayed graphically for ease of characterization. All results were statistically analyzed by comparing the average results for an induction or treatment group with the average result for its control group at the same time point using analysis of covariance (“ANCOVA”) followed by the Hochberg's procedure and those with statistical significance had ρ<0.05 unless otherwise noted.

It should be appreciated that it is not necessary to use ANCOVA followed by Hochberg's procedure to statistically analyze data in a method of this invention. Alternative statistical analyses are known that may be used such as ANCOVA without Hochberg's procedure, analysis of variance (“ANOVA”) with Hochberg's procedure, ANOVA without Hochberg's procedure, t-test with Hochberg's procedure, and t-test without Hochberg's procedure.

It should also be appreciated that the IL-6 and IL-6sR used as described in all of the below examples was the commercially available recombinant human IL-6 and recombinant human IL-6sR purchased from R&D Systems, Minneapolis, Minn.

It should also be appreciated that prior to injection of hrIL-6, hrIL-6sR, or hrIL-6 plus hrIL-6sR in all of the below examples, rats were anesthetized with 5% volume/volume (“v/v”) isoflurane gas until knocked down and maintained with 2% v/v isoflurane. Rats became fully conscious about 5 minutes after isoflurane administration was discontinued.

It should also be appreciated that in all of the below examples, the cytokine vehicle comprised 0.5% HPMC plus 0.2% Tween 80 in water (“HPMC/Tween 80”).

It will be shown below in Method Examples 1 and 2 that administration of a test compound with pain alleviating properties will inhibit (IL-6)-, (IL-6sR)-, and (IL-6 plus IL-6sR)-induced pain. However, before describing alleviation of this induced pain, it should be appreciated that the relative timing of oral administration of a test compound and the intra-articular injection of may need to be adjusted depending upon the time to peak plasma concentration of the particular compound being tested if achieving a particular result is desired.

In general there has been observed a longer time to peak plasma concentration of test compound after oral administration compared to the time to adequate pain induction following intra-articular injection of IL-6, IL-6sR, or a combination of IL-6 and IL-6sR in the IL6 Rat. Accordingly, typically a test compound (dissolved or finely suspended in a test compound vehicle) is administered orally prior to intra-articular injection of IL-6, IL-6sR, or a combination of IL-6 and IL-6sR. A preferred timing of the intra-articular injection of IL-6, IL-6sR, or a combination of IL-6 and IL-6sR in the IL6 Rat is about 1 hour before a predicted peak plasma concentration of the test compound. The 1 hour post intra-articular injection of the IL-6, IL-6sR, or IL-6 plus IL-6sR is long enough to allow for satisfactory pain induction in a joint and short enough to allow for rapid screening of test compounds for pain alleviation activity.

For illustrative example, if the time to peak plasma concentration is 2 hours post oral administration of a test compound, a preferred timing of an intra-articular injection of the IL-6, IL-6sR, or IL-6 plus IL-6sR is about 1 hour post administration of the test compound. Alternatively, if the time to peak plasma concentration is 4 hours post oral administration of a test compound, a preferred timing of an intra-articular injection of the IL-6, IL-6sR, or IL-6 plus IL-6sR is about 3 hours post administration of the test compound.

Nevertheless, it should be appreciated that it is not necessary for the purposes of determining pain alleviation according to the method of the instant invention to characterize pain in the affected joint of a test animal at about the time of peak plasma concentration of test compound. The pain may be characterized prior to the time of peak plasma concentration of test compound or subsequent to such time. In some circumstances, such as in cases where a test compound may be suspected of having a delayed onset of action, the pain may be characterized well past the time of peak plasma concentration. In such cases, test compound may be administered, and the intra-articular injection of the IL-6 or IL-6 plus IL-6sR repeated as needed, over a prolonged period of time of from several hours to several weeks. In other circumstances, such as in cases where an early answer (e.g., yes or no) to whether or not a particular test compound alleviates pain, the pain may be characterized prior to the time of peak plasma concentration of test compound. It should be appreciated that it is well known in the pharmacology art how an artisan would determine an acceptable timing to characterize pain to answer a particular question of pain alleviating activity of a test compound.

It should be appreciated that it is well known in the pharmacology art how an artisan would determine the time of peak plasma concentration of test compound post oral administration in a particular animal species. Typically, test compound is orally administered to a separate group of the animals, and blood plasma is drawn at known time points T such as, for example, T=0 hour (i.e., just prior to administration of test compound), 30 minutes, 1 hour, 2 hours, 3 hours, 6 hours, 12 hours, and 24 hours, and frozen. The frozen plasma samples are then thawed, and the thawed plasma samples are analyzed by high performance liquid chromatography (“HPLC”) against standard solutions having known concentrations of test compound to determine both the time point of peak plasma concentration and the actual plasma concentration of test compound, typically expressed as milligram of test compound per milliliter of plasma (mg/mL).

The ability of IL-6 or a combination of IL-6 and IL-6sR was demonstrated as described below in Demonstration Examples 1 and 2, respectively. The ability of IL-6sR or a combination of heat inactivated IL-6 and IL-6sR to induce pain in a knee joint was demonstrated as described below in Demonstration Example 3. The ability of the invention method to identify arthritic pain alleviating agents and characterize known pain alleviating agents was determined as described further below in Method Examples 1 and 2.

### DEMONSTRATION EXAMPLE 1

(IL-6)-Induced Joint Pain:

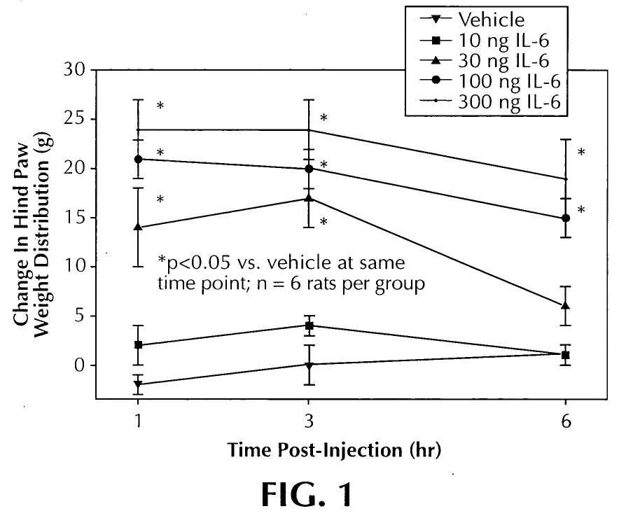

Hind-paw weight differentials between the right joint and the left joint of male Wistar rats (150 g) were determined for 30 rats with the incapacitance tester. Rats were then randomly divided into five groups of 6 rats each, a control group and four inducement groups. Rats were anesthetized with 5% v/v isoflurane gas until knocked down and maintained with 2% v/v isoflurane gas. At Time 0, each rat was injected with 50 μL of PBS into a control knee. In addition, each rat in the control group was injected with 50 μL of PBS into the contralateral test knee and each rat in each of the four inducement groups was injected with 50 μL of PBS containing 10 ng, 30 ng, 100 ng, or 300 ng, respectively, of hrIL-6. The injections were done through the patellar ligament into the knee joint space. The isoflurane gas was discontinued. At Times 1, 3, and 6 hours, pain in each rat was characterized by measuring hind paw weight bearing differentials with the incapacitance tester, and the results were statistically analyzed. The results are shown in FIG. 1.

As mentioned above, FIG. 1 (“FIG. 1”) is a line graph that shows the effect on rat hind paw weight distribution of an intra-articular injection through the patellar ligament into the joint space of certain amounts (10, 30, 100, or 300 ng) of hrIL-6 dissolved in 50 μL of PBS versus injection of 50 μL of PBS alone. In FIG. 1, it can be seen that induction groups having injection of 100 ng or 300 ng of hrIL-6 alone produced a statistically significant change in hind paw weight differential at 1, 3, and 6 hours post injection compared to the vehicle control group. The induction group having injection of 30 ng of hrIL-6 alone produced a statistically significant change in hind paw weight differential at 1 and 3 hours post injection compared to the vehicle control group, but the change became non-significant at 6 hours post injection. The induction group having injection of 10 ng of hrIL-6 alone did not produce a statistically significant change in hind paw weight differential at 1, 3, or 6 hours post injection compared to the vehicle control group. The data displayed in FIG. 1 demonstrate that intra-articular injection into a rat knee joint of more than 10 ng of hrIL-6 only will induce pain in the joint. The data also demonstrate that hrIL-6 will induce joint pain in a rat.

### DEMONSTRATION EXAMPLE 2

(IL-6 Plus IL-6sR)-Induced Joint Pain:

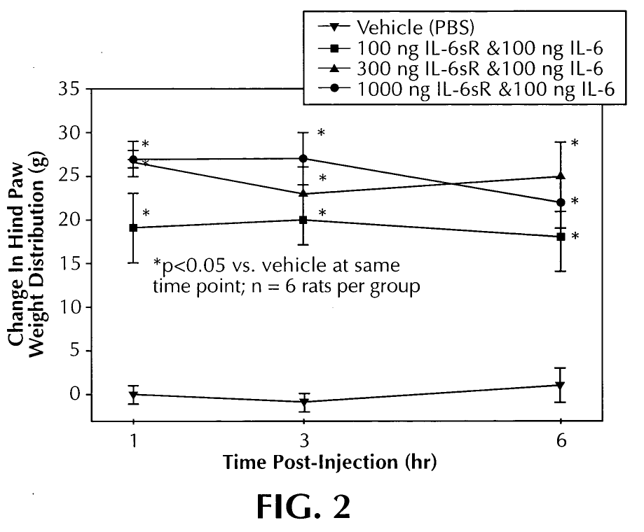

Hind-paw weight differentials between the right joint and the left joint of male Wistar rats (150 g) were determined for 24 rats with the incapacitance tester. Rats were then randomly divided into four groups of 6 rats each, a control group and three inducement groups. Rats were anesthetized with 5% v/v isoflurane gas until knocked down and maintained with 2% v/v isoflurane gas. At Time 0, each rat was injected with 50 μL of PBS into a control knee. In addition, each rat in the control group was injected with 50 μL of PBS into the contralateral test knee and each rat in each of the three inducement groups was injected with 50 μL of PBS containing 100 ng of hrIL-6 and 100, 300, or 1000 ng, respectively, of hrIL-6sR, wherein the hrIL-6 and hrIL-6sR were premixed about 0.25 hours prior to injection. The injections were done through the patellar ligament into the knee joint space. The isoflurane gas was discontinued. At Times 1, 3, and 6 hours, pain in each rat was characterized by measuring hind paw weight bearing differentials with the incapacitance tester, and the results were statistically analyzed. The results are shown in FIG. 2.

As mentioned above, FIG. 2 (“FIG. 2”) is a line graph that shows the effect on rat hind paw weight distribution of an intra-articular injection through the patellar ligament into the joint space of a combination of 100 ng hrIL-6 and certain amounts of hrIL-6sR (100, 300, or 1000 ng) dissolved in 50 μL of PBS versus injection of 50 μL of PBS alone. In FIG. 2, it can be seen that all induction groups produced a statistically significant change in hind paw weight differential at 1, 3, and 6 hours post injection compared to the vehicle control group. The data displayed in FIG. 2 demonstrate that intra-articular injection into a rat knee joint of 100 ng of hrIL-6 in combination with 100, 300, or 1000 ng of hrIL-6sR will induce pain in the rat joint.

### DEMONSTRATION EXAMPLE 3

(IL-6)-, (IL-6sR)-, (IL-6 Plus IL-6sR)-, and (Heat Inactivated IL-6 Plus IL-6sR)-Induced Joint Pain:

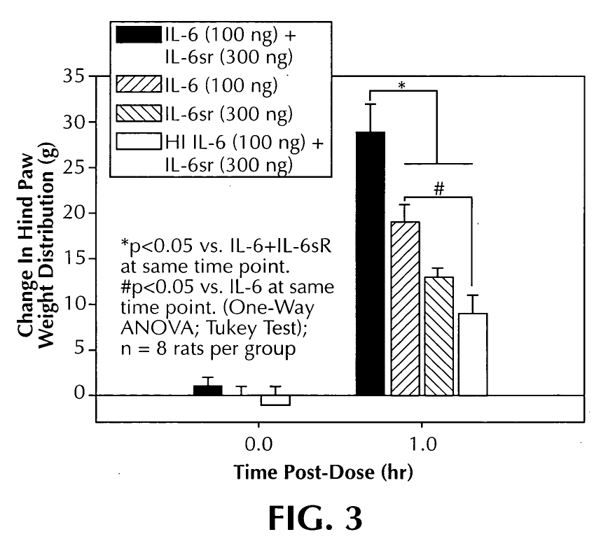

Hind-paw weight differentials between the right joint and the left joint of male Wistar rats (150 g) were determined for 32 rats with the incapacitance tester. Rats were then randomly divided into four groups of 8 rats each. Rats were anesthetized with 5% v/v isoflurane gas until knocked down and maintained with 2% v/v isoflurane gas. At Time 0, each rat was injected with 50 μL of PBS into a control knee. In addition, each rat was injected in the contralateral knee with 50 μL of PBS containing a combination of 100 ng of hrIL-6 and 300 ng of hrIL-6sR, wherein the hrIL-6 and hrIL-6sR were premixed about 0.25 hours prior to injection, 100 ng of hrIL-6 alone, 300 ng of hrIL-6sR alone, or a combination of 100 ng of heat inactivated hrIL-6 (“HI hrIL-6,” a solution of human IL-6 in PBS was heated at 100° C. for from 10 to 20 minutes and cooled to room temperature) and 300 ng of hrIL-6sR, wherein the heat inactivated hrIL-6 and hrIL-6sR were premixed about 0.25 hours prior to injection. The injections were done through the patellar ligament into the knee joint space. The isoflurane gas was discontinued. At 1-hour post injection, pain in each rat was characterized by measuring hind paw weight bearing differentials with the incapacitance tester, and the results were statistically analyzed. The results are shown in FIG. 3.

As mentioned above, FIG. 3 (“FIG. 3”) is a bar graph that shows the effect on rat hind paw weight distribution before (Time 0 hour) and after (Time 1 hour) an intra-articular injection through the patellar ligament into the joint space of a combination of 100 ng of hrIL-6 and 300 ng of hrIL-6sR, 100 ng of hrIL-6 alone, 300 ng of hrIL-6sR alone, or a combination of 100 ng of heat inactivated hrIL-6 and 300 ng of hrIL-6sR, each dissolved in 50 μL of PBS. The data displayed for the injection of a combination of 100 ng of hrIL-6 alone, 300 ng of hrIL-6sR alone, or a combination of 100 ng of heat inactivated hrIL-6 and 300 ng of hrIL-6sR were statistically analyzed by a one-way analysis of variance (“ANOVA”) and Tukey test. The data displayed in FIG. 3 demonstrate that intra-articular injection into a rat knee joint of a combination of 100 ng of hrIL-6 and 300 ng of hrIL-6sR, 100 ng of hrIL-6 alone, or 300 ng of hrIL-6sR alone will induce pain in the joint. Further, although heat inactivation of 100 ng of hrIL-6 decreased the ability of its combination with 300 ng of hrIL-6sR to induce pain compared to a combination of untreated 100 ng of hrIL-6 and 300 ng of hrIL-6sR, nevertheless the combination having the heat inactivated human IL-6 still induced pain in the rat in a statistically significant manner.

As shown by FIGS. 1 to 3 and Demonstration Examples 1 to 3 above, IL-6 alone, IL-6sR alone, a combination of IL-6 and its soluble receptor IL-6sR, or a combination of IL-6 that has been inactivated by heat and IL-6sR each rapidly induce pain in an animal when injected intra-articularly into the animal's joint space.

### METHOD EXAMPLE 1

(IL-6)- or (IL-6 Plus IL-6sR)-Induced Arthritic Pain in Rat (“IL6 Rat”) and its Dose Response Alleviation by Rofecoxib or Naproxen:

Hind-paw weight differentials between the right joint and the left joint of male Wistar rats (150 g) were determined for 45 rats with the incapacitance tester. Rats were then randomly divided into nine groups of 5 rats each, a control group, four rofecoxib treatment groups, and four naproxen treatment groups. At Time 0, each rat in the control group was administered HPMC/TWEEN 80 test compound vehicle alone, each rat in the rofecoxib treatment groups was administered 1, 3, 10, or 30 mg/kg of rofecoxib, and each rat in the naproxen treatment groups was administered 1, 3, 10, or 30 mg/kg of naproxen. Rats were anesthetized with 5% isoflurane gas until knocked down and maintained with 2% isoflurane gas. At Time 1 hour, each rat was injected with 50 μL of PBS into a control knee. In addition, each rat in the control group was injected with 50 μL of PBS into the contralateral test knee and each rat in each of the treatment groups was injected with 50 μL of PBS containing 100 ng of hrIL-6 and 300 ng of hrIL-6sR, wherein the hrIL-6 and hrIL-6sR were premixed about 0.25 hours prior to injection. The injections were done through the patellar ligament into the knee joint space. The isoflurane gas was discontinued. At Time 2 hours, pain in each rat was characterized by measuring hind paw weight bearing differentials with the incapacitance tester, averaging the results for each group, and calculating percent inhibition of pain in the treatment groups compared to the control group using the following equation:

Percent inhibition of a change in hind paw weight distribution

$$Percent inhibition of a change in hind paw weight distribution = \{ 1 - [ \frac{( Δ {W}_{G} )}{( Δ {W}_{C} )} ] \} \times 100$$

wherein: ΔW_{C} is the hind-paw weight differential between the healthy limb and the hrIL-6+hrIL-6sR injected limb of the control animal group administered vehicle alone, as measured at time 1 hour post injection; and

ΔW_{G} is the hind-paw weight differential between the healthy limb and the hrIL-6+hrIL-6sR injected limb of a treatment animal group as measured at time 1 hour post injection.

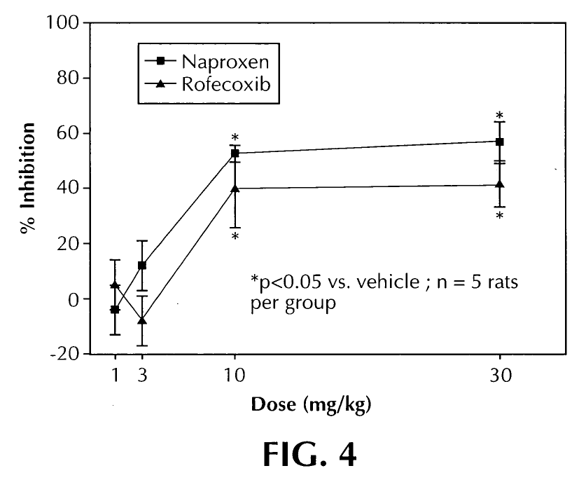

The inhibition results were then statistically analyzed. The results are shown in FIG. 4.

As mentioned above, FIG. 4 (“FIG. 4”) is a dose response line graph that shows the percent inhibition of a change in rat hind paw weight distribution following oral administration at Time 0 of 1, 3, 10, and 30 mg/kg of naproxen or rofecoxib dissolved in HPMC, followed by an intra-articular injection through the patellar ligament into the joint space at Time 1 hour of a combination of 100 ng hrIL-6 and 300 ng of hrIL-6sR dissolved in 50 μL of PBS, followed by hind paw weight distribution measurements taken at Time 2 hours (1 hour post injection), compared to a control group for oral administration of HPMC/TWEEN 80 alone. The data displayed in FIG. 4 demonstrate that both rofecoxib and naproxen each independently inhibit pain in a statistically significant manner when administered at a dose of 10 mg/kg or greater compared to administering test compound vehicle alone.

### METHOD EXAMPLE 2

(IL-6)- or (IL-6 Plus IL-6sR)-Induced Arthritic Pain in Rat (“IL6 Rat”) and its Time Course of Alleviation by Rofecoxib or Naproxen:

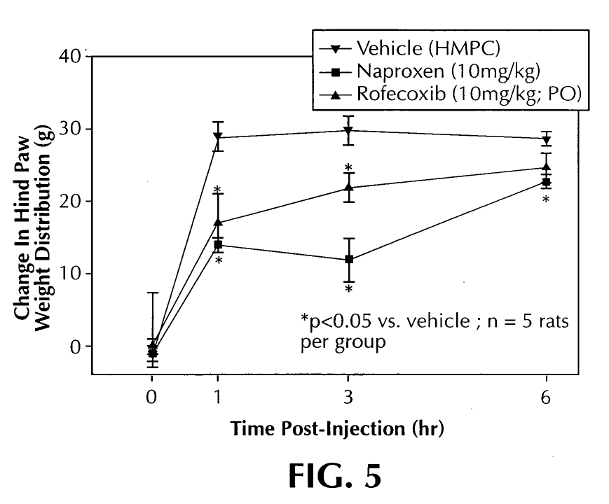

Fifteen male Wistar rats (150 g) were randomly divided into three groups of 5 rats each, a control group, one rofecoxib treatment group, and one naproxen treatment group. At Time 0, hind paw weight differentials between the right joint and the left joint for each of the rats were determined with the incapacitance tester, and each rat in the control group was administered HPMC/TWEEN 80 test compound vehicle alone, each rat in the rofecoxib treatment groups was administered 10 mg/kg of rofecoxib, and each rat in the naproxen treatment groups was administered 10 mg/kg of naproxen. Rats were anesthetized with 5% isoflurane gas until knocked down and maintained with 2% isoflurane gas. At Time 1 hour, each rat was injected with 50 μL of PBS into a control knee. In addition, each rat in the control group was injected with 50 μL of PBS into the contralateral test knee and each rat in each of the treatment groups was injected with 50 μL of PBS containing 100 ng of hrIL-6 and 300 ng of hrIL-6sR, wherein the hrIL-6 and hrIL-6sR were premixed about 0.25 hours prior to injection. The injections were done through the patellar ligament into the knee joint space. The isoflurane gas was discontinued. Additional hind paw weight differentials were determined with the incapacitance tester at Times 2, 4, and 7 hours post administration of rofecoxib or naproxen (i.e., 1, 3, and 6 hours, respectively, post injection) to characterize pain in each rat. The results were averaged for each group and then statistically analyzed. The results are shown in FIG. 5.

As mentioned above, FIG. 5 (“FIG. 5”) is a time course line graph that shows the change in rat hind paw weight distribution following oral administration at Time 0 of 10 mg/kg of naproxen or 10 mg/kg of rofecoxib dissolved in HPMC/TWEEN 80 and an intra-articular injection through the patellar ligament into the joint space at Time 1 hour of a combination of 100 ng hrIL-6 and 300 ng of hrIL-6sR dissolved in 50 μL of PBS, compared to a control group for oral administration of HPMC/TWEEN 80 alone. The rat hind paw weight distribution data were measured at Time 0, 1, 3, and 6 hours post injection or 1, 2, 4, and 7 hours post administration, respectively. The data displayed in FIG. 5 demonstrate that both rofecoxib and naproxen each independently reduced pain in a statistically significant manner when administered at a dose of 10 mg/kg compared to test compound vehicle alone at Time 1 and 3 hours post injection of hrIL-6 and hrIL-6sR and naproxen reduced pain in a statistically significant manner when administered at a dose of 10 mg/kg compared to test compound vehicle alone at Time 6 hours post injection.

The foregoing studies establish that injected IL-6, IL-6sR, or IL-6 plus IL-6sR will induce pain in a rat, mouse, or rabbit joint that can be alleviated by administering an effective amount of an analgesic compound. Further, IL-6 and/or IL-6sR from one animal species may be used in a method of this invention that employs a different animal species. The studies establish that the methods of the present invention provide a rapid, reproducible method for screening test compounds for pain alleviation or characterizing the pain alleviation effects of known analgesics.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

Having described the invention, certain aspects of the invention are hereupon claimed.

What is claimed is:

1. A method for determining alleviation by a test compound of pain in an animal, comprising:
administering a measured amount of a test compound dissolved in a test compound vehicle to each of a group of from 1 to 500 treatment animals;
administering to a hind leg knee joint of each of the treatment animals a pain inducing amount of IL-6 dissolved in a cytokine vehicle, IL-6sR dissolved in a cytokine vehicle, or of a combination of IL-6 and IL-6sR dissolved in a cytokine vehicle;
characterizing pain in the hind leg knee joint of each of the treatment animals after the administration of IL-6 dissolved in a cytokine vehicle, IL-6sR dissolved in a cytokine vehicle, or of a combination of IL-6 and IL-6sR dissolved in a cytokine vehicle by measuring differential hind paw weight distribution; and
comparing the differential hind paw weight distribution measurements for the from 1 to 500 treatment animals to a control value.

2. The method according to claim 1, further comprising the step of deriving the control value by:
administering to a hind leg knee joint of each of a group of from 1 to 500 control animals a pain inducing amount of IL-6 dissolved in a cytokine vehicle, IL-6sR dissolved in a cytokine vehicle, or of a combination of IL-6 and IL-6sR dissolved in a cytokine vehicle; and
characterizing pain in the hind leg knee joint of each of the control animals by measuring differential hind paw weight distribution.

3. The method according to claim 1, further comprising the step of deriving the control value by:
administering an amount of a test compound vehicle to each of a group of from 1 to 500 control animals;
administering to a hind leg knee joint of each of the control animals a pain inducing amount of IL-6 dissolved in a cytokine vehicle, IL-6sR dissolved in a cytokine vehicle, or of a combination of IL-6 and IL-6sR dissolved in a cytokine vehicle; and
characterizing pain in the hind leg knee joint of each of the control animals by measuring differential hind paw weight distribution.

4. The method according to claim 1, wherein the control value is derived before the test compound dissolved in test compound vehicle and the IL-6 dissolved in a cytokine vehicle, IL-6sR dissolved in a cytokine vehicle, or of the combination of IL-6 and IL-6sR dissolved in a cytokine vehicle are administered.

5. The method according to claim 1, wherein the control value is derived before the comparison of the differential hind paw weight distribution measurements.

6. The method according to claim 1, comprising:
administering an amount of a test compound vehicle to each of a group of from 1 to 500 control animals;
administering a measured amount of the test compound dissolved in a test compound vehicle to each of a group of from 1 to 50 treatment animals;
administering to a hind leg knee joint of each of the control animals and treatment animals a pain inducing amount of IL-6 dissolved in a cytokine vehicle, IL-6sR dissolved in a cytokine vehicle, or of a combination of IL-6 and IL-6sR dissolved in a cytokine vehicle;
characterizing pain in the hind leg knee joint of each of the control animals and treatment animals by measuring differential hind paw weight distribution; and
characterizing the differential of pain in the hind leg knee joints of the control animals and pain in the hind leg knee joints of the treatment animals.

7. The method according to claim 1, comprising:
administering a measured amount of a test compound vehicle to each of a group of from 1 to 500 control animals;
administering a measured amount of the test compound dissolved in a test compound vehicle to each of a group of from 1 to 50 treatment animals;
administering to a first hind leg knee joint of each of the control animals and treatment animals a pain inducing amount of IL-6 dissolved in a cytokine vehicle, IL-6sR dissolved in a cytokine vehicle, or a combination of IL-6 and IL-6sR dissolved in a cytokine vehicle;
administering to a second hind leg knee joint of each of the control animals and treatment animals a measured amount of cytokine vehicle;
characterizing pain in the first hind leg knee joint of each of the control animals and treatment animals by measuring differential hind paw weight distribution; and
characterizing the differential of pain in the first hind leg knee joints of the control animals and pain in the first hind leg knee joints of the treatment animals.

8. The method according to claim 1, wherein a combination of hrIL-6 and hrIL-6sR is administered.

9. The method according to claim 1, wherein hrIL-6 or hrIL-6sR is administered.

10. The method according to claim 1, wherein each animal is a rat, mouse, or rabbit.
